(19) United States
(12) Patent Application Publication
(10) Pub. No.: US 2015/0112415 A1
(43) Pub. Date: Apr. 23, 2015

(54) Active, Reversible Fixing of CRT Electrodes

(71) Applicant: BIOTRONIK SE & Co. KG, Berlin (DE)

(72) Inventors: Pierre Weitzig, Irrel (DE); Detmar Jadwizak, Erkner (DE); Gordon Hillebrand, Berlin (DE); Carsten Fruendt, Berlin (DE)

(21) Appl. No.: 14/495,069

(22) Filed: Sep. 24, 2014

Related U.S. Application Data
(60) Provisional application No. 61/891,921, filed on Oct. 17, 2013.

(51) Int. Cl. A61N 1/05

(52) U.S. Cl. CPC A61N 1/057; A61N 2001/058
USPC 607/116; 29/746

(57) ABSTRACT

An implantable medical electrode device includes an elongate electrode line with a tubular electrode body, having a hollow, passing from the proximal to distal ends, an electrode provided at the distal end, and an electrode connection provided at the proximal; a fixing device with a balloon catheter arrangement, having an elongate tubular hollow catheter body with an outer diameter smaller than the inner diameter of the hollow of the electrode body, and an inflatable balloon at the distal end, with an outer diameter smaller in the deflated state compared to the inner diameter of the hollow of the electrode body, and a fluid connection at the proximal end adapted to the electrode connection of the electrode body; connection means for releasable connection of the electrode line to the fixing device; and sealing means for fluid-tight closure of the catheter body of the fixing device in an inflated balloon state.

### CROSS-REFERENCE TO RELATED APPLICATIONS

This patent application claims the benefit of co-pending U.S. Provisional Patent Application No. 61/891,921, filed on Oct. 17, 2013, which is hereby incorporated by reference in its entirety.

### TECHNICAL FIELD

The present invention relates to an implantable medical electrode device and, in particular to a cardiovascular cardiac pacemaker electrode device, adapted for use in a blood vessel, comprising an elongate electrode line with a tubular electrode body, which has a hollow, passing from the proximal end to the distal end, with an inner diameter, and at the distal end of which an electrode is provided and at the proximal end of which an electrode connection is provided, and a fixing device for determining the position in the blood vessel. The present invention further relates to a fixing device for such an electrode device and also to a corresponding medical electrode implantation system.

### BACKGROUND

A basic requirement when it comes to the use and therefore the design of implantable electrode lines is the assurance of their correct position in a hollow organ or vessel of the body, in which they are to be effective. The development and constant improvement of suitable fixing devices has therefore been an intrinsic part of the technical development in this field for many years, and a large number of different fixing mechanisms are known.

Particularly in the case of what are known as CRT electrodes (electrode lines for biventricular or 3-chamber pacemakers), nubs or tines on the periphery of the distal electrode end, silicone screws or helices protruding from the electrode end, or also pre-punched J-shaped or S-shaped curvatures of the distal end portion of the electrode line are used, inter alia, as a passive fixing means, or, for example, a slitted tube compressed manually at the site of use is used as an active fixing means.

International Publication No. WO 94/07564 discloses a solution for the problem of repositionability, in which the fixing is realized by an expandable or self-expanding wire basket. Disadvantages of this design include the fact that the wires cut into the vessel wall and may therefore be very traumatic, and the fact that it cannot be explanted reversibly.

Reference is further made to U.S. Publication No. 2006/0036307. This document discloses an implantable cardiovascular cardiac pacemaker electrode device or defibrillator electrode device, which comprises an elongate, tubular electrode body and a fixing zone in the distal end region of the electrode body. The fixing zone has an outwardly closed peripheral sleeve and is used to releasably fix the electrode device in a body lumen in which it is reversibly expandable. The control of the expansion or contraction is ensured by an expansion means arranged in the fixing zone.

In addition to these features, European Patent No. EP 0 546 414 presents an electrode device in which the expansion means is formed by being acted on pneumatically or hydraulically by pressure, with the aid of which the wall of the electrode body, said wall being flexible in the fixing zone, is reversibly expandable.

European Patent No. EP 1 832 309, in the name of the Applicant, presents a further implantable medical electrode device, specifically, a cardiac pacemaker electrode device or a defibrillator electrode device, in which the fixing problem is solved to a certain extent by the provision of a stent-like, plastically deformable supporting structure in the wall of the electrode body. This supporting structure can be transferred into a statically expanded fixing state by being acted on by overpressure, and can be transferred into a contracted starting state by being acted on by negative pressure. In this case, this is a special, integrated electrode line construction of rather complex design, which cannot be explanted reversibly.

It is known from International Publication No. WO 2010/138648 to fix a guide wire in a vessel with the aid of an expandable balloon integrated in said guide wire and to then advance, via the guide wire, an electrode line in a manner known per se to the desired point of the vessel, at which the guide wire is fixed by means of the balloon.

With most conventional passive fixings, the contact pressure on the vessel wall and, therefore, the fixing in the desired position is dependent on the pre-shaping of the electrode in the distal region, as developed by the electrode manufacturer.

Pre-shapings by heat or by mechanical loading can negatively influence the helix properties in terms of long-term stability. The quality (or the contact pressures) of the pre-shapings by heat or mechanical loading fluctuates very strongly, for example, due to different wire batches. Passive fixings are often unsuitable for the large variation of vessel diameters. Fixings, such as, for example, the silicone screw and nubs, are suitable for small vessels (e.g., target veins), whereas J-bends, helices or S-curves are suitable for larger vessels (e.g., coronary sinus or outlets of the coronary sinus). As a result, the electrode cannot always be placed in its optimal position with a good stimulus threshold. The dislocation rate of a CRT electrode is additionally much higher than with right-heart electrodes, which are implanted in the atrium or in the ventricle. In addition, there is a risk that the electrode just positioned will be displaced as the guide catheter is removed (e.g., slitten).

In some cases, an unfavorable position with a poor stimulation threshold is accepted because a left-heart electrode cannot be securely fixed in its optimal target vein.

The function of a fixing, that is to say to have a certain rigidity in order to press well against the vessel wall, conflicts with flexibility, softness and maneuverability during the implantation process.

Active fixings have the large disadvantage of not being explantable. The principle lies in generating irregular thicknesses, anchorings or material agglomerations during the implantation process. The electrode is thus no longer isodiametric and the fixing elements become firmly ingrown, which makes explantation impossible. Explantation is vital, however, in the case of infections, for example.

The present invention is directed toward overcoming one or more of the above-mentioned problems.

### SUMMARY

An object of the present invention is consequently to provide an electrode device of which the functional characteristics under different conditions are improved and which, where necessary, can be explanted without difficulty.

These and further objects are achieved by an electrode device having the features of claim 1. Furthermore, a medical electrode implantation system having the features of claim 11 is provided.

The following considerations of the inventors form part of the present invention:

This fixing should drastically minimize dislocations of a placed electrode. It must only develop after the implantation and is to be greater than the electrode diameter. It has to anchor securely in coronary veins having different diameters.

This fixing is to be used optionally and can additionally be combined with, or supplemented by, any conventional CRT electrode on the market. In addition, the fixing should not impair a potential repositioning or explantation of the electrode. The fixing element in the “ineffective state” is to have a diameter smaller than the electrode line body. The fixing element is to be independent of the electrode design and of the used materials.

These considerations lead to the provision of a fixing device with a balloon catheter arrangement which has an elongate, tubular, hollow catheter body with an outer diameter that is smaller than the inner diameter of the hollow of the electrode body, an inflatable balloon at, or close to, the distal end, with an outer diameter smaller in the deflated state compared to the inner diameter of the hollow of the electrode body, and a fluid connection at the proximal end, which is adapted to the electrode connection of the electrode body. Furthermore, the provision of connection means for releasable connection of the electrode line to the fixing device and sealing means for fluid-tight closure of the catheter body of the fixing device in its state of use with an inflated balloon also form part of the present invention.

In terms of its handling, the electrode device according to the present invention is presented as follows:

In an embodiment of the present invention, a plug adapter is attached to the proximal end of the fixing device and is adapted on the inner face to receive the electrode connection of the electrode line and on the outer face for insertion into an electrode socket of an electromedical device, in particular, a cardiac pacemaker, and also for insertion of an injection cannula into the fluid connection of the catheter body. In embodiments of the present invention that are specially tailored to CRT electrodes, the outer electrode connection of the plug adapter is formed as an IS-1 or IS-4 electrode connection.

The electrode line (in principle any CRT electrode) is implanted by conventional methods. After the removal of the guide wire or of the stylet, the balloon catheter arrangement (“balloon fixing”) can be introduced. The electrode plug is inserted here into the plug adapter on the tube of the fixing balloon. With the aid of a matching cannula and hand sprayer, the balloon can be inflated with a liquid medium in order to fix the electrode. Due to the sealing means in the plug adapter, an automatic deflation of the fixing balloon is prevented. The adapter is then slid together with the electrode into the cavity of the pacemaker or defibrillator housing and is anchored.

With a possible repositioning or explantation, the adapter can be removed together with the electrode from the housing of the apparatus, and the cannula with the hand sprayer can be inserted in order to remove the liquid from the balloon and to deflate the balloon.

The plug adapter is preferably suitable for either an IS-1 plug or for an IS4 plug.

In a further embodiment of the present invention, the sealing means comprises a resilient cylindrical seal element, which is arranged concentrically with the catheter body of the balloon catheter arrangement and which, from the proximal end, can be pierced by an injection cannula in such a way that it again closes in a fluid-tight manner automatically after removal of the injection cannula.

In a further embodiment of the present invention, the connection means for releasable connection comprises adjustment means, in particular a fixing screw, which is embedded in the electrode body, is accessible from the outer periphery thereof, and is oriented radially for infiltration into the hollow of the electrode line and for engagement with the tubular catheter body of the fixing device inserted therein, the adjustment means being designed to adjust the longitudinal position of the fixing device with respect to the electrode line.

With regard to materials of the present invention, the balloon of the balloon catheter arrangement can be formed from a material from the group comprising silicone, PUR, ETFG, PTFE and PA. In addition, known and proven materials can be used for the components of the proposed electrode device, such as the respective tubes, electrodes, feed lines, etc. The fixing balloon may, for example, be filled with NaCl, silicone oil or also with X-ray contrast medium to additionally enable representation in an X-ray image; this selection is to be understood expressly, however, as being non-limiting.

In an embodiment as a modular electrode implantation system, the proposed solution provides fixing tools of different length, in particular, which allow optimal adaptation to different vessel sizes and desired electrode positions within a vessel, and more specifically beyond the adjustment possibilities provided by an individual fixing tool on account of inherent adjustment options (see further below). In this important aspect, the modular solution according to the present invention of all fixing devices integrated fixedly into an electrode line is considered to be fundamental.

With the present invention, significant advantages compared to known solutions are in any case achieved in some of the above-mentioned embodiments:

It can be used optionally for any CRT electrode obtainable on the market (that is to say the doctor can decide during the implantation process whether he/she would like to use this auxiliary tool, for example, in the case of insufficient fixing of the electrode or unfavorable anatomical conditions).

The fixing is independent of the vessel size.

The fixing is independent of the number of poles of an electrode (e.g., unipolar, bipolar and quadripolar).

The fixing does not impair the isodiametric design of the electrode.

The fixing is independent of the materials and the design of the CRT electrode.

The dislocation rate would reduce considerably.

The optimal position and, therefore, optimal stimulus threshold (therapy) of the electrode can be adjusted without difficulty.

CRT applications do not have to be interrupted because fixing of the electrode is not possible.

The proposed electrode device can be explanted in principle.

Further features, aspects, objects, advantages, and possible applications of the present invention will become apparent from a study of the exemplary embodiments and examples described below, in combination with the Figures, and the appended claims.

### DESCRIPTION OF THE DRAWINGS

Advantages and expedient features of the present invention will emerge in addition from the description of an exemplary embodiment on the basis of the Figures, in which:

FIG. 1 shows a perspective view of an exemplary embodiment of the implantable electrode device according to the present invention in a starting state,

FIG. 2 shows a perspective view of an exemplary embodiment of the implantable electrode device according to the present invention in the implanted state (fixing state),

FIG. 3 shows a perspective view of a detail of the electrode device shown in FIGS. 1-2,

FIG. 4 shows a perspective view of the device part shown in FIG. 3 with fitted injection cannula, and

FIG. 5 shows a detailed view (longitudinal sectional illustration) of the device part shown in FIGS. 3-4, together with the end of the injection cannula.

### DETAILED DESCRIPTION

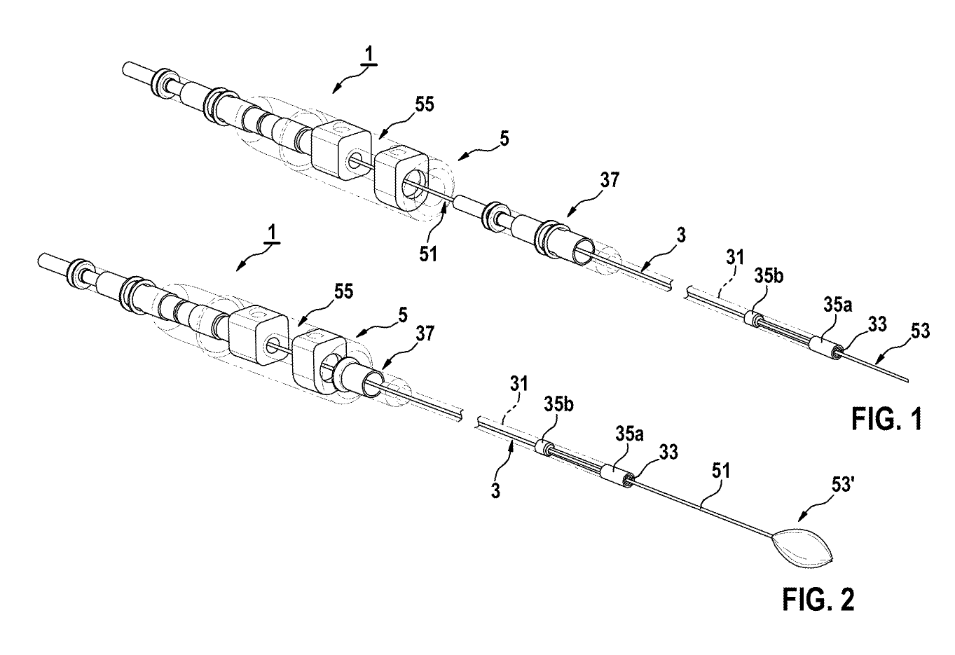

FIGS. 1-2 each show an implantable medical electrode device 1, which comprises an electrode line 3 formed as what is known as a CRT electrode, and a fixing device 5, which is formed as a balloon catheter arrangement and is manufactured separately, but is connected in use to the electrode device 1. Since the structure of the electrode line 3 is known and details are not of significance for the explanation of the present invention, the individual parts/portions are not identified in greater detail in the Figures and will not be described in greater detail hereinafter. It is merely noted that the electrode line 3 has an elongate tubular electrode body 31 with a hollow 33 passing from the proximal end to the distal end, and that two electrodes 35a, 35b are provided at, or close to, the distal end of the electrode body, and, for example, an IS-1 or IS4 standard plug 37 is provided at the proximal end. (Alternatively, the electrode line may also have just one of the up to four electrodes.)

The fixing device 5 comprises an elongate tubular catheter body 51 with an inflatable balloon portion 53 (FIG. 1) or inflated balloon 53′ (FIG. 2) at the distal end and a plug adapter 55, of which the inner structure will be described in greater detail hereinafter, at the proximal end. In FIG. 1, the fixing device 5 is removed distally slightly with respect to the electrode line 3, whereas in FIG. 2 both components of the electrode device 1 are shown in the stated connected to one another for use and with the balloon 53′ already inflated.

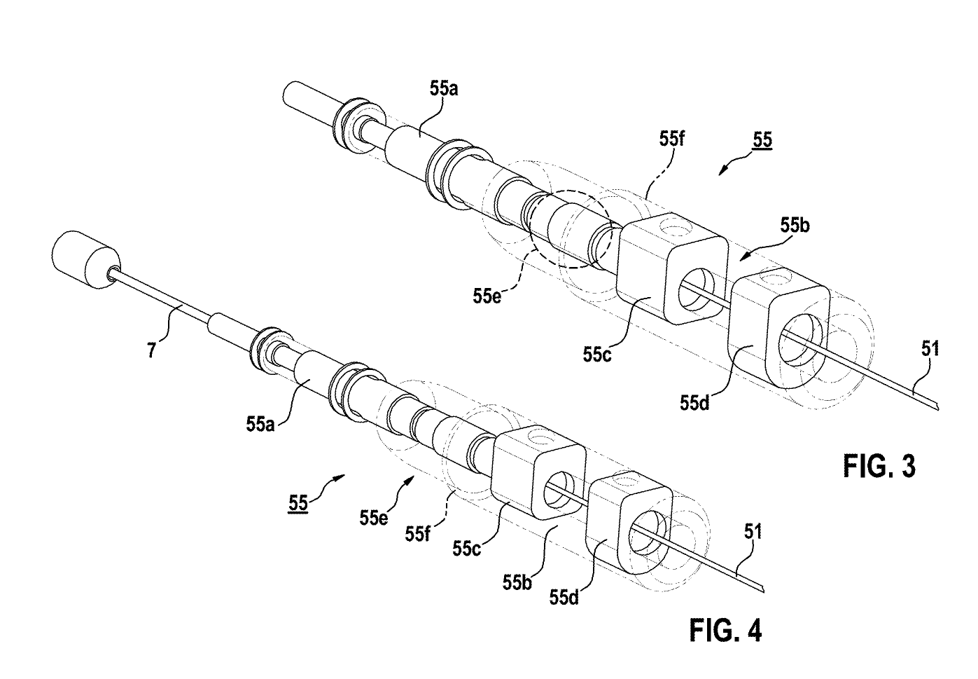

FIGS. 3-4, in two illustrations of a detail of the electrode device 1, show the plug adapter 55 of the fixing device 5 in greater detail and, more specifically in FIG. 4, with a proximally fitted injection cannula 7 for filling the balloon (not illustrated here) of the fixing device. The plug adapter 55, at its proximal end, has a connection plug 55a of identical design to the connection plug 37 of the electrode line 3, that is to say of the IS-1 type in the present case, for example. At its distal end, a corresponding standard socket (IS-1 socket) 55b is provided, for example. This socket portion comprises two contact blocks 55c, 55d, each with a set screw (not denoted separately) for contacting and fixing the plug portion of the inserted electrode line (not shown here). A sealing unit 55e is arranged between the plug portion 55a and the socket portion 55b, and all mentioned parts are combined in an integral plug adapter housing 55f.

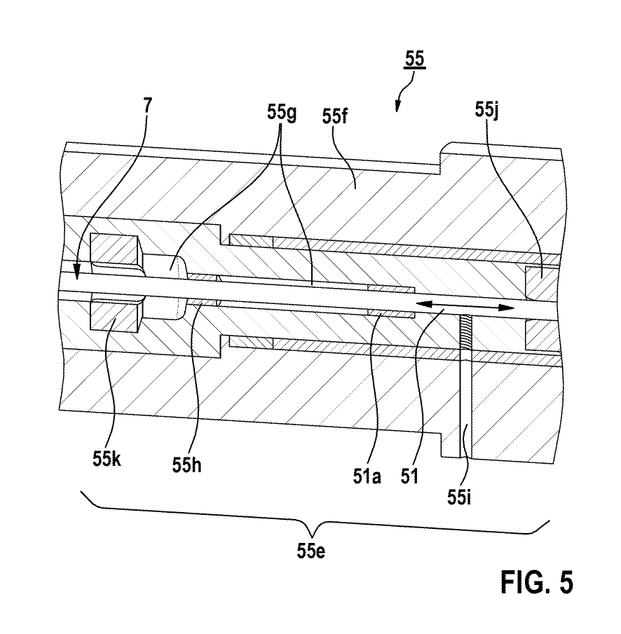

FIG. 5 shows a perspective longitudinal sectional illustration of the internal structure of the plug adapter 55, especially of the sealing unit 55e, in even greater detail. A two-part filler chamber 55g, which comprises a proximal chamber portion of relatively large diameter but shorter length, and a distal chamber portion of smaller diameter but relatively large length (not denoted separately), is arranged centrally in the sealing unit 55e. The distal chamber portion is dimensioned such that the catheter tube 51 fits therein in a liquid-tight manner and is longitudinally displaceable therein. Both at the distal end of the balloon tube 51 and also between the two portions of the filler chamber 55g, a tube stop 51a and 55h is provided respectively.

In the adapter housing 55f, a thread 55i for a set screw (not shown) for fixing the longitudinal position of the catheter tube 51 with respect to the plug adapter 55 is provided distally of the distal end of the filler chamber 55g. The fixing screw is intended for the longitudinal fixing of the position of the entire tool on the catheter body (balloon tube). The position (spacing) of the fixing balloon starting from the tip (distal end) of the electrode can thus be varied.

Further distally from this thread 55i, a distal seal element 55j is attached in the adapter body, and a central seal element is also provided at the proximal entry of the filler chamber 55g and is denoted by numeral 55k. In this case, this is a seal element that can be pierced by the cannula 7 and is self-closing after removal of the cannula 7 and that is designed such that it can permanently hold the liquid pressure in the filler chamber/catheter/balloon system of the fixing device 5, and can thus ensure the outer dimensions of the balloon and, hereby in turn the fixing effect thereof, in a lasting manner.

The embodiments of the present invention are not limited to the above-described examples and highlighted aspects, but can also be implemented in a large number of modifications within the scope of the capabilities of a person skilled in the art.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range.

I/We claim:

1. An implantable medical electrode device adapted for insertion into a blood vessel, comprising:
an elongate electrode line with a tubular electrode body, which has a hollow, passing from a proximal end to a distal end, with an inner diameter, and at the distal end of which one or more electrodes are provided, and at the proximal end of which an electrode connection is provided;
a fixing device with a balloon catheter arrangement, which has an elongate tubular hollow catheter body with an outer diameter that is smaller than the inner diameter of the hollow of the electrode body, and an inflatable balloon at, or close to, the distal end, with an outer diameter smaller in a deflated state compared to the inner diameter of the hollow of the electrode body, and a fluid connection and electrode connection at the proximal end, which is adapted to the electrode connection of the electrode body;
connection means for releasable connection of the electrode line to the fixing device; and
sealing means for fluid-tight closure of the catheter body of the fixing device in its state of use with an inflated balloon.

2. The electrode device as claimed in claim 1, wherein a plug adapter is attached to the proximal end of the fixing device and is adapted on its inner face to receive an electrode connection of the electrode line and on its outer face for insertion into an electrode socket of an electromedical device, and is also adapted for insertion of an injection cannula into the fluid connection of the catheter body.

3. The electrode device as claimed in claim 1, wherein the sealing means comprise a resilient cylindrical seal element, which is arranged concentrically with the catheter body of the balloon catheter arrangement and which, from the proximal end thereof, can be pierced by an injection cannula in such a way that it again closes in a fluid-tight manner automatically after removal of the injection cannula.

4. The electrode device as claimed in claim 1, wherein adjustment means, which is embedded in the electrode body, is accessible from the outer periphery thereof, and is oriented radially for infiltration into the hollow of the electrode line and for engagement with the tubular catheter body of the fixing device inserted herein, is provided to adjust the longitudinal position of the fixing device with respect to the electrode line.

5. The electrode device as claimed in claim 2, wherein the outer electrode connection of the plug adapter is formed as an IS-1 or IS4 electrode connection.

6. The electrode device as claimed in claim 1, wherein the balloon of the balloon catheter arrangement is formed from a material from the group comprising silicone, PUR, ETFG, PTFE and PA.

7. The electrode device as claimed in claim 1, wherein the implantable medical electrode device comprises a cardiovascular cardiac pacemaker electrode device.

8. The electrode device as claimed in claim 4, wherein the adjustment means comprises a fixing screw.

9. A fixing device for forming an electrode device as claimed in claim 1.

10. An electrode line, designed to form an electrode device as claimed in claim 1.

11. A medical electrode implantation system, comprising:
an electrode device as claimed in claim 1; and
a fluid injection arrangement with an injection needle, which has an outer diameter adapted to the proximal fluid connection of the balloon catheter arrangement.
